(19) United States
(12) Patent Application Publication
(10) Pub. No.: US 2002/0006402 A1
(43) Pub. Date: Jan. 17, 2002

(54) Administering IgE antagonists during pregnancy to ameliorate allergic diseases in the offspring

(75) Inventors: David Anderson, Houston, TX (US); David Thomas, Houston, TX (US)

Correspondence Address: Eric Mirabel, Tanox, Inc., 10301 Stella Link # 110, Houston, TX, 77025-5497, US

(73) Assignee: Tanox, Inc.

(21) Appl. No.: 09/759,658

(22) Filed: Jan. 12, 2001

Related U.S. Application Data
(60) Provisional application No. 60/175,092, filed on Jan. 7, 2000.

(51) Int. Cl. A61K 39/395

(52) U.S. Cl. 424/131.1

(57) ABSTRACT

The invention relates to IgE antagonists, including monoclonal antibodies, and their use in ameliorating asthma and allergic diseases in offspring of mothers treated during pregnancy with such antagonists. The preferred IgE antagonists do not induce release of the mediators of allergy. One example of such IgE antagonists are anti-IgE antibodies which bind to secreted IgE, to membrane IgE on the surface of IgE-producing B cells, but not to IgE bound to the Fc∈RI on the surface of basophils or mast cells. Preferably, these antibodies also do not bind to IgE bound to Fc∈RII receptors. It is also preferable if these antibodies have human IgG1 or IgG3 constant regions, as well as further human portions, if desired.

### FIELD OF INVENTION

The invention relates to IgE antagonists, including monoclonal antibodies, and their use in ameliorating asthma and allergic diseases in offspring of mothers treated during pregnancy with such antagonists.

### BACKGROUND OF THE INVENTION

Immunoglobulin E (IgE) is one class of immunoglobulin (or “antibody”) molecule. IgE is present in human serum in lower concentrations than the other immunoglobulins: IgG, IgM, IgA, and IgD. IgE is thought to have a role in protection against parasites, but has never been definitively established as playing a necessary, or even a beneficial role, at least in developed countries where parasite infections are not a significant problem. IgE is well known as the mediator of immediate-type hypersensitivity allergic reactions, including allergic rhinitis (“hay fever”), extrinsic asthma, and food and drug allergies.

In IgE-mediated allergic reactions, IgE, after it is secreted by B cells, binds through its Fc portion to the Fc∈RI receptors, which are present on the surface of basophils, mast cells and Langerhans cells. If the IgE bound to the surface of these cells now contacts and binds an allergen, this causes a cross-linking of the bound IgE molecules and hence the underlying receptors, and triggers the release of pharmacologic mediators, such as histamine, serotonin, leukotrienes and the slow-reacting substance of anaphylaxis. These mediators cause the pathologic manifestations of allergic reactions.

A particular class of anti-IgE antibodies has been developed to treat allergic diseases. These antibodies bind to secreted IgE, but not to IgE attached to the Fc∈RI receptors. When these anti-IgE antibodies are administered intemally, they bind to IgE and neutralize it, thereby preventing its binding to either Fc∈RI or Fc∈RII receptors, the latter receptor being present on B cells and other cell types as well. These anti-IgE antibodies also bind to IgE which is attached to the membrane of IgE-producing B cells (the “membrane form of IgE”). By doing so, they may further aid in down-regulating or eliminating, through antibody dependent cellular cytotoxicity (“ADCC”) or complement mediated cytolysis, the IgE-producing B cells, and therefore, reduce the levels of secreted IgE. Because they do not bind to IgE attached to the Fc∈RI, however, they do not cause cross-linking and do not themselves result in release of pharmacologic mediators of allergy.

It has been shown that such anti-IgE antibodies can lower IgE levels, in both animal models and human clinical trials. See Come et al., J. Clin. Invest. 99, No. 5, 879-887 (1997). Such anti-IgE antibodies also demonstrated efficacy in treating allergic rhinitis and extrinsic asthma in several human clinical trials. See Come et al., id.; Boulet et al., Am J. Respir. Crit. Care Med., 155: 1835-1840 (1997); Fahy et al., J. Respir. Crit. Care Med., 155: 1828-1834 (1997); Milgrom et al., New Eng. J. Med. 341:1966-1973 (1999). No clinical trials of these anti-IgE antibodies have been performed where pregnant mothers are treated to determine if such treatment reduces incidence or severity of asthma in their offspring. However, there are reports that reducing a pregnant woman's exposure to allergens or controlling her allergic reactions may prevent the development of allergic disease in the child. See Ann Allergy Asthma Immunol. 83(5):426-430 (1999). It is believed that treatment with anti-IgE or other IgE antagonists, which control allergic responses, could reduce the incidence or severity of allergic diseases, including asthma, in offspring of treated mothers.

### SUMMARY OF INVENTION

The invention includes IgE antagonists, including monoclonal antibodies, for use in ameliorating asthma and allergic diseases in offspring of mothers treated during pregnancy with such antagonists. The IgE antagonists and monoclonal anti-IgE antibodies of the invention function to reduce free IgE levels in a patient. The preferred monoclonal anti-IgE antibodies of the invention bind to secreted IgE but not to IgE bound to the Fc∈RI receptors, which receptors are present on basophils, mast cells, or Langerhans cells. The preferred anti-IgE antibodies preferably also bind to membrane IgE, and thereby aid in down-regulating or eliminating IgE-producing B cells, leading to further reduction in secreted IgE levels. These anti-IgE antibodies preferably do not bind to IgE bound to the low affinity Fc∈RII receptors. If the antibodies of the invention did bind to IgE bound to the Fc∈RII receptors, they could cause the destruction or down-regulation of B cells producing other classes of immunoglobulins, or destruction or down-regulation of other cell types, which would be undesirable.

Monoclonal anti-IgE antibodies can be modified to be less immunogenic and more suitable for human administration by techniques including chimerization, humanization (through CDR-grafting), or otherwise. Another class of antibodies with reduced immunogenicity are fully human antibodies. These can be produced in transgenic animals or synthesized from single chain fragments of human antibodies produced through phage display library technology. Preferably, the monoclonal anti-IgE antibodies of the invention have a human IgG1 or IgG3 constant heavy chain region, as such regions are known to mediate ADCC and complement mediated cytolysis, thereby aiding in elimination of IgE-producing B cells.

The IgE antagonists of the invention are likely to be most effective when internally administered, such as by intravenous, intramuscular, or subcutaneous injection. They can also be internally administered by oral ingestion, in a suitable carrier which is not subject to digestive degradation, or through the alveoli of the lung by an inhaler.

Another method of administering the IgE antagonists of the invention is using a synthetic or recombinant peptide or an anti-idiotype antibody, which include all or part of the sequence of IgE, to induce endogenous production of anti-IgE antibodies. A related method is to use a gene therapy vector to induce endogenous production of anti-IgE antibodies. The gene encoding suitable anti-IgE antibodies is administered to the mother by suitable means. It is incorporated in the cells and programs them to produce the anti-IgE antibodies.

One could also generate anti-Fc∈RI antibodies by administering a peptide corresponding to the FcERI sequence. Such antibodies may have the same effect as anti-IgE when administered to pregnant mothers.

### DESCRIPTION OF MAKING AND USING THE INVENTION

1. Making the Various Embodiments of the Invention

Chemical or biological entities suitable for use as IgE antagonists can be selected and screened by a number of methods, including using assays similar to those used to screen TES-C21, described below. In essence, one would screen first for those that bound to secreted IgE, and then, from that group, those that did not induce release of pharmacologic mediators of allergy would be selected. A number of different assays, well known to those in the art, could be used to accomplish this.

In one specific embodiment, the monoclonal anti-IgE antibodies used with this invention are produced by continuous (immortalized), stable, antibody-producing cell lines. The preferred antibody-producing cell lines are hybridoma and transfectoma cell lines. However, they can be any cell lines which contain and are capable of expressing functionally rearranged genes which encode the antibodies (or fragments) of interest. Lymphoid cells which naturally produce assembled immunoglobulin are preferred.

Hybridoma cells which produce the specific antibodies used with this invention can be made by the standard somatic cell hybridization technique of Köhler and Milstein, Nature 256:495 (1975) or similar procedures employing different fusing agents. Briefly, the procedure is as follows. The monoclonal anti-IgE antibodies are produced by immunizing an animal with human IgE or IgE-producing B cells, or peptidic segments of human IgE (secretory or membrane), which are identified as including the epitope of interest, which is in the Fc region of IgE. Peptides can be synthesized or produced by recombinant DNA technology and, for enhanced antigenic effect, conjugated to a carrier protein, such as keyhole limpet hemocyanin. Following immunization, lymphoid cells (e.g., splenic lymphocytes) are obtained from the immunized animal and fused with immortalizing cells (e.g., myeloma or heteromyeloma) to produce hybrid cells. The hybrid cells are screened to identify those which produce the desired anti-IgE antibody by following the screening methods described below in detail.

It is preferred that the antibodies be either human or substantially human, to reduce or eliminate the human anti-mouse (HAMA) response. The murine antibody portions of a murine antibody could themselves trigger an allergic response, or the HAMA response against such portions could reduce the effectiveness of the treatment.

A technique for producing human antibodies is through production in transgenic mice. Briefly, this approach involves disruption of endogenous murine heavy and kappa light chain loci, followed by construction of heavy and light chain transgenes containing V, D, J segments, and C genes of human origin. These are then introduced by pronuclear microinjection using human transgenes. The mice are then cross-bred to generate the human antibody producing strains. This technique is describe in more detail in, among other references, U.S. Pat. No. 5,569,825 (incorporated herein by reference). The technology may be available under license from Medarex, Inc. (Annandale, New Jersey).

Another alternative for solving antigenicity problems is to produce fully human antibody fragments, for example, the single chain Fv region, by the phage display library methodology. Briefly, this involves amplification of the human V gene repertoire from bone marrow, blood and tonsil samples by polymerase chain reaction (“PCR”), followed by preparation of separate libraries containing heavy and light chain (both κand λ) chain V genes. These separate fragments are then assembled into a single chain Fv for display on the surface of phage, where the desired fragments can be readily screened. References describing this technique in more detail include U.S. Pat. No. 5,565,332 (incorporated by reference) and European Patent No. 0 589 877 B1. The technology may also be available under license from Cambridge Antibody Technology Limited, Melboum, England.

Production of antibodies in rodents, especially mice, is a very well established procedure. One established method to reduce the murine portions of the anti-IgE antibodies is to produce them in a rodent system, and convert them into chimeric rodent/human antibodies or CDR-grafted humanized antibodies by established techniques. Chimeric antibodies can be produced as described, for example, in U.S. Pat. No. 4,816,397 (incorporated by reference). The making of humanized antibodies is described, among other references, in U.S. Pat. Nos. 5,693,762; 5,693,761; 5,225,539 (both incorporated by reference), and in WO 89/06692 and WO 92/22653. As another alternative, one can made a Delmmunised™ antibody. In Deimmunised™ antibodies, T and B cell epitopes have been eliminated, as described in International Patent Application PCT/GB98/01473. They have reduced immunogenicity when applied in vivo.

One example of an anti-IgE antibody of the invention (designated TES-C21) and its chimeric mouse-human form (designated TESC-2) is described in International Application W092/17207. The screening protocols (described below) for TES-C21 and TESC-2 can be applied to other anti-IgE antibodies to yield antibodies of the invention suitable for chimerization or humanization through CDR-grafting. The hybridoma cell lines producing TES-C21 are available from the American Type Culture Collection (“ATCC”), Rockville, Md. under Accession No. 11134, and those producing TESC-2 are on deposit under Accession No. BRL 10706.

A humanized version of the murine antibody TES-C21 was made, as described in detail in Australian Patent No. 675449, granted May 25, 1997. Similar procedures can be followed to produce other humanized anti-IgE antibodies. Several transfectomas producing humanized anti-IgE antibodies suitable for use with the invention are available from the ATCC under the following accession numbers: 11130; 11131; 11132; 11133. An anti-IgE antibody similar to that produced from the transfectoma deposited under accession number 11131 is among those with potential for full clinical development. Another humanized antibody suitable for use in the invention is E25 (rhuMAb-E25), produced by Genentech, Inc. This antibody is described in Presta et al., J. ImmunoL 151:2623-2632 (1993).

### EXAMPLE I

### Production and Screening of TES-C21 and TESC-2

TES-C21 and TESC-2 were produced and screened as follows. Briefly, male Balb/c mice were immunized several times with polyclonal human IgE from sera (provided by Ventrex). The IgE was combined with a suitable adjuvant. Mice were sacrificed after the last injection of immunogen and the spleens were removed for preparing single cell suspensions for fusion with myeloma cells. The spleen cells were fused with Sp2/0 cells using a fusion mixture of polyethylene glycol 1450 (Kodak), CMF-PBS and DMSO. DMEM was added after the cell suspensions were combined.

The hybridomas resulting from the fusion were then screened by enzyme-linked immunosorbent assay (ELISA) against human IgE bound to an Immulon 2 plate. One of these hybridomas produced TES-C21.

TES-C21 was further screened, by ELISA, for specificity for human IgE, and for non-reactivity with IgG, IgM, IgA, IgD, human serum albumin, transferrin or insulin. TES-C21 bound equally well to various human IgE molecules. TES-C21 bound to the IgE-secreting cell lines SKO-007, U266 and SE44 in a dose-dependent manner, indicating binding to human membrane IgE. But TES-C21 did not bind to human B cell lines bearing surface IgM, IgD, IgG, or IgA, or to a T cell line, or to the parent murine cell line of SE44, or to a murine cell line secreting chimeric human IgG. TES-C21 also does not bind to IgE present on high affinity Fc&RI receptors or on low affinity FcERII receptors. These receptors are present on a wide variety of cell types. It also did not induce histamine release from freshly prepared human blood basophils, on which the Fc∈R are armed with IgE. At 10 μg/ml TES-C21 was able to inhibit completely the binding of 1 μg of IgE to Fc∈RII.

To generate TESC-2, Sp 2/0 cells were co-transfected with the variable regions of TES-C21 H and L-chains, and human γ1 and μ constant regions, and aliquoted into 96 well plates for selection. Supernatants were screened for secretion of human IgG which bound to human IgE. The transfectoma cells were adapted to growth in serum-free medium. TESC-2 was then purified from medium of confluent cultures using an immobilized protein A column.

TESC-2 and TES-C21 bind equally well to IgE bound to microtiter plates. This was demonstrated as follows. Immulon 2 plates were coated with gp120 peptide-ovalbumin conjugate and IgE-SE44 was bound to the immobilized antigen. TES-C21 or TESC-2 at various concentrations were added. Binding was detected using either horseradish peroxidase (“HRP”),-conjugated goat antimouse IgG (for TES-C21) or HRP-goat antihuman IgG, Fc (for TESC-2).

It was determined that TESC-2 and TES-C21 also have the same relative affinity for IgE bound to microtiter plates. Immulon 2 plates were coated with gp120 peptide-ovalbumin conjugate and IgE-SE44 was bound to the immobilized antigen. TES-C21 and TESC-2 at various concentrations were added and preincubated for 1 hour before adding 0.22 μg/ml of biotinylated TES-C21. Binding of biotinylated TES-C21 was detected using horseradish peroxidase-conjugated streptavidin.

TESC-2 and TES-C21 also were shown to bind equally to IgE-producing cells. This was demonstrated by incubating such cells at 2×10⁶ cells/100 μ1 PBS-1% goat serum at various antibody concentrations at 0° for 30 minutes. Binding of TES-C21 was detected using FITC-goat (Fab')₂ antimouse IgG. Binding of TESC-2 was detected using FITC-goat (Fab')₂ antihuman IgG. Binding was quantitated by fluorescence flow cytometry using a Coulter Epics V. The FITC intensity gate was set to yield 10%±0.5% positive cells in the absence of primary immunoglobulins.

It was found that neither TES-C21 nor TESC-2 bound to IgE which was bound to low affinity IgE receptors. The possibility that TESC-2 recognized IgE complexed with CD23 was studied using cells of an IgG-secreting human lymphoblastoid line, IM-9. The presence of CD23 on IM-9 cells was confirmed by their strong staining with anti-Leu 20, a MAb specific for CD23. IM-9 cells were incubated with 5 to 10 μg/ml of human IgE, washed, and then incubated with biotin-labeled TESC-2 or a positive control anti-IgE Mab TE-19, followed by FTIC-streptavidin and analyzed by flow cytometry.

Both chimeric TESC-2 and murine TES-C21 were shown to inhibit binding of IgE to Fc∈RII. The antibodies were preincubated at various concentrations with 20 μg IgE-SE44 for 1 hour at 370 before addition of IM-9 cells bearing Fc∈RII. Binding of IgE to cells was detected using biotinylated TES-19 and FITC-streptavidin and quantitated by fluorescence flow cytometry.

To negate the possibility that immune complexes of TESC-2 and IgE, formed during their preincubation in these experiments, were binding to cells but yielding false negatives, it was confirmed that these immune complexes also did not bind to Fc∈RII, using biotin-labeled TESC-2 or FITC goat anti-human IgE (with TES-C21).

Neither TESC-2 nor TES-C21 induces histamine release from freshly prepared human blood basophils on which the Fc∈R are armed with IgE. Due to the variable release of mediators from basophils of different donors, the antibodies were examined at multiple concentrations on basophil preparations from more than 50 individual donors. No induction of histamine release by TESC-2 or TES-C21 was observed.

To address the possibility that TES-C21 might bind to basophils but not induce cross-linking of the receptors to induce histamine release, a secondary antibody was used for crosslinking. Since anti-human IgG alone can induce histamine release, only the murine antibody TES-C21 was used in these experiments. The crosslinking goat antimouse IgG enhances histamine release induced by suboptimal concentrations of control anti-IgEs. However, TES-C21 did not induce histamine release even under these very permissive conditions.

TESC-2 was further tested to determine whether it could block the binding of IgE to Fc∈RI receptors, and whether immune complexes of IgE and TESC-2 would bind to these receptors. To determine whether TESC-2 inhibits the binding of human IgE to Fc∈RI, human peripheral blood basophils that had been depleted of IgE by treatment at low pH were reloaded or sensitized with SE44 -derived chimeric IgE reactive to a peptide antigen. Functional binding of SE44 IgE was tested by histamine release induced by the polyvalent R15K peptide-ovalbumin conjugate to which the variable region of IgE-SE44 binds. Preincubation of IgE-SE44 with TESC-2 inhibited IgE binding to Fc∈RI. Binding of SE44 was also inhibited when basophils were incubated with another IgE (PS) before exposure to IgE-SE44 . It may be assumed that immune complexes of TESC-2 and IgE were formed during the preincubation and these also did not cause the release of histamine. The experimental conditions and the results of these experiments are summarized below in Table 1.

TABLE 1
Inhibition of IgE Binding to High-Affinity
IgE Receptors by TESC-2

| | Net Histamine Release of (% of total) | |
|---|---|---|
| Conditions for Basophil | Challenge with R15K | Challenge |
| Loading with IgE-SE44 | Peptide-Ovalbumin | with Anti-IgE |
| IgE-SE44 was not preincubated | 37 | 66 |
| with TESC-2 | | |
| IgE-SE44 was preincubated with | 3 | 68 |
| TESC-2 | | |
| IgE-SE44 was preincubated with | 0 | 63 |
| IgE-PS | | |

These studies have also been performed, and similar results obtained, with the CDR-grafted version of TES-C21 referenced above.

2. Using the Antibodies of the Invention for Ameliorating Allergic Disease in the Offspring of Pregnant Mothers

Prior to commercial availability, the IgE antagonists, or antibodies, of the invention must be subjected to human clinical trials to confirm their safety and efficacy. A sample protocol for such a clinical trial would be to start with a number pregnant patients having asthma or allergic rhinitis or another allergic disease, and administer some active IgE antagonist and some a placebo. The offspring would then be monitored to determine if those born from the treated mothers had a lower incidence or severity of allergic disease than those born from the women receiving the placebo.

In this protocol, if anti-IgE is selected as the IgE antagonist to be used, patients would receive intravenous or subcutaneous injections of 50 to 300 mg of anti-IgE at weekly, bi-weekly or monthly intervals during pregnancy, or for a period of 9 months.

Additional studies would investigate alternative dosing schedules and dosing intervals. The IgE antagonists of the invention, administered by any acceptable route and for any acceptable time period, are expected to have a substantial beneficial effect for offspring of mothers suffering from allergic disease.

The foregoing description, terms, expressions and examples are exemplary only and not limiting. The invention includes all equivalents of the foregoing embodiments, both known and unknown. The invention is limited only by the claims which follow and not by any statement in any other portion of this document or in any other source.

What is claimed is:

1. A method of inhibiting allergic disease in an offspring comprising administering an IgE antagonist to the mother while she is pregnant.

2. The method of claim 1 wherein the IgE antagonist is an anti-IgE antibody which binds to secreted IgE but not to basophils.

3. The method of claim 2 wherein the IgE antagonist binds to secreted IgE and membrane-bound IgE.

4. The method of claim 3 wherein the IgE antagonist does not bind to IgE which is bound to the Fc∈RII receptor.

5. The method of any of claims 2 to 4 wherein the anti-IgE antibody is a monoclonal antibody.

6. The pharmaceutical composition of claim 5 wherein the anti-IgE antibody is a chimeric, humanized (CDR-grafted), or human antibody.

7. The pharmaceutical composition of claim 6 wherein the anti-IgE antibody has a human IgGI or IgG3 heavy chain constant region.

8. The method of claim 1 wherein the IgE antagonist is a fragment of an antibody, including a Fab, F(ab)′2 or a single chain antibody.

9. A method of inhibiting allergic disease in an offspring comprising administering a composition which induces expression of IgE antagonists to the mother while she is pregnant.

10. The method of claim 9 wherein the composition includes a peptide, an anti-idiotype antibody or a gene encoding an anti-IgE antibody or a fragment thereof.

11. A method of inhibiting allergic disease in an offspring comprising administering a composition which induces expression of anti-Fc∈RI antibodies to the mother while she is pregnant.

12. The method of claim 11 wherein the composition includes a peptide encoding a portion of the sequence of IgE.

13. The method of claim 11 wherein the composition includes a gene encoding an anti-IgE antibody or a fragment thereof.
